(19) United States
(12) Patent Application Publication
(10) Pub. No.: US 2024/0156795 A1
(43) Pub. Date: May 16, 2024

(54) METHODS AND COMPOSITIONS FOR THE TREATMENT OF DRUG RESISTANT TOPICAL INFECTIONS

(71) Applicant: Lankenau Institute for Medical Research, Wynnewood, PA (US)

(72) Inventors: Melvin Reichman, West Chester, PA (US); George C. Prendergast, Penn Valley, PA (US)

(21) Appl. No.: 18/550,919

(22) PCT Filed: Mar. 17, 2022
(86) PCT No.: PCT/US2022/020756

§ 371 Date: Sep. 15, 2023

Related U.S. Application Data
(60) Provisional application No. 63/162,675, filed on Mar. 18, 2021.
(60) Provisional application No. 63/117,718, filed on Nov. 24, 2020.

(51) Int. Cl. A61K 31/4418; A61K 9/00; A61K 31/155; A61K 31/22; A61K 31/405; A61K 31/4178; A61K 33/18; A61K 47/14; A61K 47/20; A61P 17/00; A61P 31/10

(52) U.S. Cl. CPC A61K 31/4418; A61K 9/0014; A61K 31/155; A61K 31/22; A61K 31/405; A61K 31/4178; A61K 33/18; A61K 47/14; A61K 47/20; A61P 17/00; A61P 31/10

(57) ABSTRACT

Compositions and methods for the treatment of topical infections such as onychomycosis are disclosed.

### BACKGROUND OF THE INVENTION

The treatment of onychomycosis continues to be a common and intractable problem in adults. Topical treatments have very poor efficacy [1], while oral agents have significant risk of hepatotoxicity [2]. Disease recurrences are frequent and can become the source of more widespread disease [3]. For example, onychomycosis represents an important risk factor for the development of diabetic foot syndrome and foot ulcers leading to amputations [4]. Though rare, toenail infection can develop other complications, including systemic infection in high-risk patients [5]. The commonly held explanation for the poor efficacy of topical toenail fungus treatments is that they are unable to diffuse past the nail barrier to penetrate deep into the nail matrix. However, oral antifungal drugs also have limited efficacy and, moreover, have significant risk of liver damage [2]. Thus, there is considerable unmet medical need for improved onychomycosis treatments. Onychomycosis is a common fungal infection of the nail seen clinically most frequently in toenails. Onychomycosis in the toenail is seen frequently in the podiatry clinic where it presents not only a difficult physical problem but also an emotional concern to patients and their caregivers. The prevalence of this condition is 6-8% in the general adult population, but far higher among diabetics, athletes, the elderly, and patients undergoing chemotherapy.

While not serious at first for most people, infected nails can become thick and misshapen over time, causing social stigma and pain, making it difficult to walk when wearing shoes. Onychomycosis occurs in 10% of the general population, 20% of persons older than 60 years, and 50% of those older than 70 years, compared to 0.7% in patients younger than 19 years of age [6]. Further, men are up to three times more likely to have onychomycosis than women, though the reasons for this gender difference are not clear [5]. Changing demographic characteristics such as the relative aging of the population, the increasing prevalence of diabetes and peripheral vascular disease, extensive use of broad-spectrum antibiotics, and more widespread iatrogenic immunosuppression are likely to increase the prevalence for onychomycosis [7]. Regardless of the drug prescribed by the clinician, long treatment durations may extend to a full year. Complete cure, defined as clinical cure (nail clearing) plus mycological cure (negative microscopy and culture), is unattainable for the vast majority of patients.

Antifungal agents in wide use for treating localized dermatophytic infections include the imidazoles (efinaconazole, ketoconazole, econazole, and oxiconazole), the allylamines (naftifine and terbinafine), and the pyridine, ciclopirox olamine [8]. The first (in 2000) FDA-approved topical drug for toenail fungus was Penlac® (generic name is ciclopirox olamine, or ‘ciclopirox’). Its pharmacology is poorly understood, but the non-specific inhibitory mechanism is believed to proceed via iron chelation [9]. Sold around the world under many brand names, it has ≤12% efficacy for complete clinical cure. The recently approved topical Jublia® (efinaconazole, a triazole inhibitor of 14α-demethylase) has a complete clinical cure rate of ≤18%. Kerydin® (tavarborole, an inhibitor of fungal leucyl-tRNA synthetase), was also FDA-approved in 2014 also has a poor efficacy for complete clinical cure (≤10%). Despite the limited drug efficacies, patient and physician preference is generally for topical agents as first-line drugs due to hepatotoxic risks of oral treatments. Hepatotoxicity is a particular concern in elderly patients or with co-occurring disorders such as diabetes, hypertension, and cancer, where polypharmacy will contraindicate oral antifungals. Due to the high potential for persistence of some pathogens within the nail unit after visible clearance of infection, relapse and re-infection are common and can occur in many patients [3,10].

Thus, curative topical therapies with low risk and cost remain highly desired.

### BRIEF DESCRIPTION OF THE DRAWINGS

FIG. 1 shows ciclopirox enhances HIF levels in B6 cells. Cells derived from B6 HIF-luciferase reporter mice were assayed ex vivo to detect the stabilization of HIF1 protein. Values are means of duplicate determinations ±SD.

FIG. 2 shows ciclopirox inhibits IDO1. IDO1 activity in U937 cells was assessed by spectrophotometric measurement of kynurenine formation in the medium at least 24 hours post-induction with IFN-γ. Values are means of duplicate determinations ±SD.

FIG. 3 shows a study design overview.

FIG. 4 shows treatment of a skin Staphylococcus infection on a patient's foot. Upper panel-pre-treatment; Lower panel-post-treatment.

### SUMMARY OF THE INVENTION

In accordance with one aspect, the provided compositions and methods for inhibiting, treating, and/or preventing topical infections, particularly drug resistant topical infections, are provided. In certain embodiments, the topical infection is onychomycosis. In certain embodiments, the topical infection is onychomycosis. In certain embodiments, the composition comprises a) at least one antimicrobial agent; b) at least one immune enhancer or wound healing agent; and c) a carrier. The composition may further comprise: a) at least one immunomodulatory agent such as an IDO/TDO inhibitor or agent that enhances regenerative healing, such as a HIF1 activator, including an agent with both these activities [bifunctional]; b) at least one broad-spectrum antiseptic. The composition may further comprise at least one penetrant, and/or keratolytic agent, such as ethyl pyruvate, DMSO, salicylate or urea, etc. The composition may further comprise at least one wound healing agent such as a HIF-½ α activator. In certain embodiments, the composition comprises iodine or chlorhexidine, and ethyl pyruvate. In certain embodiments, the composition comprises ciclopirox, iodine or chlorhexidine, and ethyl pyruvate. In certain embodiments, the components are together in a single composition or contained separately as individual units or compositions. The methods provided comprise applying the composition(s) topically, e.g., to the skin or nail of a subject.

### DETAILED DESCRIPTION OF THE INVENTION

Present clinical practices for treating infections like onychomycosis focus on eradicating a single microbe species. Thus, single antifungal drugs are used to treat nail infections—usually with very limited success—against an assumed primary fungal infection. However, drug-resistant topical infections, including onychomycosis, can manifest due to a combination of pathogenic infections and a local microenvironment (e.g., in the nail for onychomycosis) that is immunosuppressed. This immunosuppression prevents the clearance of the infection. This combination (e.g., which may be termed a ‘mal-microbiome’) resists treatment of the underlying pathogenic fungus with antifungal drugs alone. In contrast to previous therapeutic methods, formulations provided herein kill the pathogens while simultaneously boosting the patient's immune response to accelerate clearing of the infection.

The poor efficacy of current treatments for drug-resistant skin or nail infections (onychomycosis) may also reflect poor drug penetration into the skin or the nail plate. Particularly in the latter setting, the thickness and compact construction of the nail make it a formidable barrier to entry of topical agents, the concentration of which can drop up to 1000-fold from the cornified surface to the inner bed underneath. The human nail plate includes three layers: the dorsal and intermediate layers of the nail and the ventral layer comprising the nail bed. The dorsal layer is a few cell layers thick and consists of hard keratin. This layer is the main barrier to diffusion of therapeutics into and through the nail plate. The intermediate layer is also rich in keratin but behaves somewhat differently than the dorsal layer with regard to diffusion. The ventral layer consists of a soft hyponychium where fungal infections take hold beneath the nail plate. Achieving an effective drug concentration in the ventral layer is important for effective treatment of onychomycosis.

In addition to poor drug penetration, all of the causative pathogens and host defense mechanisms involved in drug-resistant topical infections like onychomycosis are not being effectively treated by current standards of care. Present clinical practices for treating onychomycosis focus on eradicating a single causative agent namely the fungus, by administering a given antifungal drug, albeit usually to abysmally low cure rates of ˜10%. However, as explained above, a drug-resistant skin infection or onychomycosis is not necessarily caused only by pathogenic microbes (e.g., fungus), but also due to a locally immunosuppressed microenvironment which allows the infection to take hold, grow and prevents clearance. This adverse synergy between a pathogen and a patient's suppressed immune system may be referred to as a malmicrobiome (MMB). Further, each of the components of the MMB should be addressed for an effective cure. Accordingly, to attack the MMB effectively, 1) the pathogenic driving components of the infection should be attacked; and also 2) simultaneously the local immune tolerance in the infected niche microenvironment should be relieved. The immunosuppression may be two-pronged including ‘extrinsic’ features from the pathogen (e.g., drug resistance) as well as ‘intrinsic’ features from the host (e.g., due to aging, diabetes, chemotherapy, or other disorders that blunt immune competency in clearing pathogens).

Herein, compositions and methods for treating, inhibiting, and/or preventing topical infections, particularly drug resistant topical infections in a subject are provided. In a particular embodiment, the topical infection is onychomycosis. In a particular embodiment, the infection is on or in the skin. In a particular embodiment, the drug resistant topical infection is onychomycosis present in a toenail. In certain embodiments, the infection is a diabetic foot ulcers, bedsores, sepsis, or an opportunistic infection. In a particular embodiment, the subject is an athlete, elderly individual, diabetic patient, or a subject undergoing chemotherapy for treating cancer, autoimmune disease, organ transplantation or a subject otherwise immune tolerant or suppressed.

In a particular embodiment, the compositions provided herein comprise a) at least one antimicrobial (including antifungals or pan-antiseptics) and b) at least one immunomodulatory agent. In a particular embodiment, the composition is for treating onychomycosis. In a particular embodiment, the compositions provided herein comprise a) an antiseptic and b) an immunomodulatory agent. In a particular embodiment, the composition comprises at least two antifungals that each have distinct mechanisms of action to exploit drug synergy in clearing a MMB infection. The composition may also comprise a pharmaceutically and/or cosmetically acceptable carrier or solvent to improve penetration through the nail bed for the pharmacological agents. A broad-spectrum antimicrobial agent (e.g., a pan-disinfectant or pan-antiseptic) may be used in place of the more selectively acting antifungal(s). For example, the composition may comprise at least one pan-antiseptic (broadly effective against at least fungi) and at least one immunomodulatory agent to boost the subject's immune response for helping clear the pathogen. The compositions may further comprise at least one wound-healing agent. In some formulations, a single agent might serve two functions, for example, a single agent can have both anti-microbial and immune stimulation activities. The compositions may also comprise at least one penetrating agent (penetrant), which also might have another function, including immune enhancement. The compositions provided herein may also comprise cosmetic agents such as coloring agents and fragrances that are pharmacologically inert. While compositions provided herein are described as a single composition comprising all of the components, also provided are multiple compositions wherein each composition comprises one or more components (but less than the entirety of the components). The multiple compositions may be mixed prior to use or may be applied sequentially or simultaneously to the nail for treatment.

The term “antifungal” refers to a substance that inhibits fungal growth and/or replication (i.e., fungistatic) and/or kills fungi (i.e., fungicidal). Examples of antifungals include, without limitation:
polyene antifungals (e.g., nystatin, amphotericin B, pimaricin, hamycin, natamycin);
azole antifungals such as:
   imidazoles (e.g., clotrimazole, miconazole, ketoconazole, econazole, tioconazole, oxiconazole, sertaconazole, sulconazole, luliconazole, butoconazole);
   triazoles (e.g., itraconazole, fluconazole, voriconazole, fosfluconazole, efinaconazole, terconazole, hexaconazole, isavuconazole, albaconazole, posaconazole);
   thiazoles (e.g., abafungin);
allyamine antifungals (e.g., naftifine, terbinafine, amorolfine, butenafine);
morpholine antifungals (e.g., amorolfine);
antimetabolite antifungals such as
   pyrimidine analogues (e.g., 5-fluorocytosine (flucytosine));
   aminoacyl tRNA synthetase inhibitors (e.g., tavaborole);
β-glucan synthase inhibitors (e.g., echinocandins, anidulafungin, caspofungin, micafungin);
mafenide (mafenide acetate);
undecylenic acid;
tolnaftate;
haloprogin; and
ciclopirox (ciclopirox olamine)

In a particular embodiment, the antifungal is selected from the group consisting of itraconazole, terbinafine, ciclopirox, and fluconazole.

“Antiseptics” are antimicrobial substances that are applied to living tissue/skin to reduce the possibility of infection, sepsis, or putrefaction. Antiseptics are generally distinguished from antibiotics by the latter's ability to be transported through the lymphatic system to destroy bacteria within the body, and from disinfectants, which destroy microorganisms found on non-living objects. Some antiseptics are true germicides, capable of destroying microbes (bactericidal), while others are bacteriostatic and only prevent or inhibit their growth. By continued exposure to antibiotics, bacteria may evolve to the point where they are no longer harmed by these compounds. Bacteria can also develop a resistance to certain antiseptics, but the effect is generally less pronounced.

Common antiseptics include but are not limited to: alcohols; chlorhexidine (gluconate); hydrogen peroxide; iodine (including tinctures and polymeric formulations (e.g., povidone-iodine); octenidine dihydrochloride; polyhexanide; and hypochlorite sodium.

As used herein, the term “penetrant” refers to agents or compounds capable of penetrating the outer layers of the nail and/or agents or compounds capable of enhancing the permeability of the nail. Chemical penetration through the nail relies upon water solubility and molecular size. Penetrants have been studied to increase diffusion of effective chemicals into/through the human nail plate, including but not limited to agents such as DMSO. Examples of penetrants that also have keratolytic actions include, without limitation, ibuprofen, salicylic acid, benzoic acid, methyl pyruvate, urea, and ethyl pyruvate, among others.

The term “antimicrobial” refers to a substance that kills or inhibits the growth of microbes such as bacteria, mycobacteria, parasites, fungi, yeast, viruses, or other microscopic organisms. Examples of antimicrobial agents (e.g., a pan-disinfectants) which are effective against at least fungi and bacteria include, without limitation: iodine (e.g., polymer iodine, tincture of iodine, etc.), chlorhexidine, hypochlorite, acetic acid, and hydrogen peroxide.

The term “immunomodulatory agent” refers to an agent that induces an immunomodulatory effect or alteration (i.e., immunostimulation) as measured, for example, by a variety of immunoassays well known in the art. In a particular embodiment, the immunomodulatory agent is an immunostimulant. Examples of immunomodulatory agents include, without limitation: indoleamine dioxygenase (IDO) inhibitors, and immune checkpoint inhibitors including but not limited to PD-1 and CTLA4 antibodies.

The term “IDO inhibitor” refers to an agent capable of inhibiting the activity of indoleamine 2,3-dioxygenase (IDO), particularly IDOL and thereby reversing IDO-mediated immunosuppression. An IDO inhibitor may be a competitive (reversibly inhibits IDO enzyme activity at the catalytic site), noncompetitive (reversibly inhibits IDO enzyme activity at a non-catalytic site), or irreversible (irreversibly destroys IDO enzyme activity, for example, by forming a covalent bond with the enzyme) IDO inhibitor. An IDO inhibitor may be an inhibitor of the IDO immune pathway. IDO inhibitors include, without limitation: D-tryptophan, D-tryptophan analogs (e.g., indoximod, 1-methyl-DL-tryptophan, 1-methyl-D-tryptophan, β-(3-benzofuranyl)-DL-alanine, beta-(3-benzo[b]thienyl)-DL-alanine, 6-nitro-L-tryptophan, methylthiohydantoin-DL-tryptophan), ethyl pyruvate, beta-carbolines (e.g., norharmane), rosmarinic acid, Cox-2 inhibitors (e.g., celecoxib), pyridoxal isonicotinoyl hydrazone, pyrrolidine dithiocarbamate, necrostatin-1, ebselen, brassinin analogs, exfoliazone, chandrananimycin A, indole 3-carbinol, 3,3′-diindolylmethane, 5-Br-4-Cl -indoxyl 1,3-diacetate, 9-vinylcarbazole, 5-bromo-DL-tryptophan, NLG-0919 (1-cyclohexyl-2-(5H-imidazo[5,1-a]isoindol-5-yl)ethanol), and 5-bromoindoxyl. Examples of IDO inhibitors are also provided, for example, Qian et al. (RSC Adv. (2016) 6:7575-7581) and U.S. Pat. Nos. 7,705,022; 7,714,139; 8,008,281; 8,389,568; 9,174,942; 8,722,720; 8,748,469; and 9,260,434. The foregoing references are incorporated by reference herein, particularly with regard to the IDO inhibitors provided therein. In a particular embodiment, the IDO inhibitor is D-tryptophan, 1-methyl-tryptophan, or methylthiohydantoin-tryptophan, or agents that chelate iron, an obligatory cofactor for IDO and other mono- and deoxygenating enzymes (for example, proline hydroxylation domain enzymes, or PHDs).

In certain embodiments, the “IDO inhibitor” refers to an agent that blocks or inhibits the expression, induction, activity, and/or signaling of one or more of IDO1, IDO2, or TDO, including, without limitation, the following compounds or a pro-drug, salt, and/or any therapeutically effective formulation of:
Indoximod (1-methyl-D-tryptophan, 1MT, NLG-8189)
1-methyl-L-tryptophan
a racemic mixture of 1-methyl-D-tryptophan and 1-methyl-L-tryptophan
Epacadostat (INCB024360; Incyte; Wilmington, DE; described in Liu et al. (2010) Blood 115(17):3520-3530; Koblish et al. (2010) Mol. Cancer Ther., 9(2):489-498))
Navoximod (NLG-919, GDC-0919, RG6078; NewLink Genetics/Genentech),
Indoximod prodrug NLG802
BMS-986205/ONO-7701 (F001287, Hunt et al., AACR 2017, Abstract 4964),
PF-06840003/EOS200271 (EOS, Wythes et al, SITC 2016, Abstract 253),
A compound of the formula, 1-R-D-tryptophan or 1-R-L-tryptophan, wherein R is a C1-C12 alkyl;
MK-7162/IOM2983 (Merck and Co.)
LY3381916 (Lilly)
KHK2455 (Kyowa Hakko Kirin)
HTI-1090/SHR9146 (Hengrui Therapeutics, Inc)
DN1406131 (De Novo Pharmatech)
RG70099 (Roche/CuraDev)
Roxyl-WL (Xu et al. J Enzyme Inhib Med Chem. 2018; 33(1): 1089-94.)
TPST-8844 (Tempest Therapeutics)
Ethyl pyruvate
AMG-1 (Amgen), as described in Smith J R et al, Novel indoleamine 2,3-dioxygenase-1 inhibitors from a multistep in silico screen, Bioorganic & Medicinal Chemistry, 20(3): 1354-1363 1 Feb. 2012, incorporated by reference herein;
methylthiohydantoin-DL-tryptophan (MTH-Trp),
β-(3-(β)-DL-alanine,
β-(3-benzo(b)thienyl)-DL-alanine,
6-nitro-L-tryptophan,
indole 3-carbinol,
3,3′-diindolylmethane,
epigallocatechin gallate,
5-Br-4-C1-indoxyl 1,3-diacetate,
9-vinylcarbazole, acemetacin,
5-bromo-DL-tryptophan,
5-bromoindoxyl diacetate,
Naphthoquinone-based,
S-allyl-brassinin,
S-benzyl-brassinin,
5-Bromo-brassinin,
Phenylimidazole-based,
4-phenylimidazole,
Exiguamine A
NSC401366
beta-lapachone (3,4-dihydro-2,2-dimethyl-2H-naphthol[1,2-b]pyran-5,6-dione, Flick et al. (2013) Int. J. Tryp. Res. 6:35-45) (ARQ 761; ArQule, now owned by Merck);
DX-03-12 and other compounds described in Wen, H. et al, Design and Synthesis of Indoleamine 2,3-Dioxygenase 1 Inhibitors and Evaluation of Their Use as Anti-Tumor Agents, Molecules 2019, 24, 2124; doi:10.3390/molecules-24112124, incorporated by reference herein;
Compounds described in patent WO2014/186035 and US Patent Publication No. US 2018/030026 (Curadev); and/or
Compounds described in WO2014/081689 and U.S. Pat. No. 9,499,497 (Vertex Pharmaceuticals).

Still other suitable inhibitors are identified in Wang, X-X et al, Recent advances in the discovery of indoleamine 2,3-dioxygenase 1 (IDO1) inhibitors, MedChemComm, and grouped as tryptophan derivatives, inhibitors with an imidazole, 1,2,3-triazole or tetrazole scaffold, inhibitors with quinone or iminoquinone, N-hydroxyamidines.

Still other examples of small molecule IDO1 inhibitors are provided, without limitation, in PCT/US2014/022680 (e.g., tricyclic compounds related to imidazoisoindoles; compounds of Formulas I-V), PCT/US2012/033245 (e.g., fused imidazole derivatives; compounds of Formula I or II), PCT/US2010/054289 (e.g., imidazole derivatives; compounds of Formulas I-VIII), PCT/US2009/041609 (e.g., compounds of Formulas I-VIII), PCT/US2008/57032 (e.g., napthoquinone derivatives; compounds of Formula I, II, or III), PCT/US2008/085167 (e.g., compounds of Formulas I-XLIV), PCT/US2006/42137 (e.g., compounds of Formula I), PCT/US2006/017983 (e.g., compounds of Formula I), PCT/US2004/005155 (e.g., phenyl-TH-DL-trp (3-(N-phenyl-thiohydantoin)-indole), propenyl-TH-DL-trp (3-(N-allyl-thiohydantoin)-indole), and methyl-TH-DL-trp (3-(N-methyl-thiohydantoin)-indole)), PCT/US2004/005154 (e.g., compounds of Formula I or II), U.S. Pat. No. 7,705,022 (e.g., compounds of Formula I), U.S. Pat. No. 8,008,281 (e.g., phenyl-TH-DL-trp (3-(N-phenyl-thiohydantoin)-indole), propenyl-TH-DL-trp (3-(N-allyl-thiohydantoin)-indole), and methyl-TH-DL-trp (3-(N-methyl-thiohydantoin)-indole)), U.S. Pat. No. 7,714,139 (e.g., compounds of Formula I or II), U.S. Patent Application Publication No. 20140066625 (e.g., fused imidazole derivatives; compounds of Formula I or II), U.S. Patent Application Publication No. 20130177590 (e.g., N-hydroxyamidinoheterocycles; compounds of Formulas I-III), U.S. Patent Application Publication No. 20140023663 (e.g., 1,2,5-oxadiazoles; compounds of Formula I), U.S. Patent Application Publication No. 20080146624 (e.g., amidines; compounds of Formulas I or II), U.S. Patent Application Publication No. 20080119491 (e.g., amidinoheterocycles; compounds of Formulas I-IV), U.S. Patent Application Publication No. 20080182882 (e.g., N-hydroxyamidinoheterocycles; compounds of Formula I), U.S. Patent Application Publication No. 20080214546 (e.g., N-hydroxyamidinoheterocycles; compounds of Formula I), U.S. Patent Application Publication No. 20060258719 (compounds of Formula I), Banerjee et al. (2008) Oncogene 27:2851-2857 (e.g., brassinin derivatives), and Kumar et al. (2008) J. Med. Chem., 51:1706-1718 (e.g., phenyl-imidazole-derivatives). In a particular embodiment, the IDO1 inhibitor is a prodrug (see, e.g., U.S. Patent Application Publication No. 20170022157 and U.S. Provisional Application No. 62/555,726). All references are incorporated by reference herein, particularly for the IDO1 inhibitors provided therein.

In a particular embodiment, the IDO1 inhibitor is ethyl pyruvate (Muller, et al. (2010) Cancer Res. 70:1845-1853) or gleevec (imatinib, Balachandran et al. (2011) Nat. Med. 17:1094-1100). In a particular embodiment, the IDO1 inhibitor (e.g., inhibitor of downstream signaling pathway) is 1-methyl-tryptophan, particularly 1-methyl-D-tryptophan (indoximod, NLG-8189; 1-methyl-D-tryptophan; NewLink Genetics), including salts and prodrugs (U.S. Patent Application Publication No. 20170022157) or a racemic mix comprising the same.

In a particular embodiment, immune checkpoint inhibitors are used in place of the above immunomodulatory agents or in combination with the above immunomodulatory agents. Examples of immune checkpoint inhibitors include, without limitation: PD-1 inhibitors (e.g., antibodies, particularly monoclonal antibodies, immunologically specific for PD-1 such as pembrolizumab (Keytruda®) and nivolumab (Opdivo®)); PD-L1 inhibitors (e.g., antibodies, particularly monoclonal antibodies, immunologically specific for PD-L1 such as atezolizumab (Tecentriq®)); and CTLA-4 inhibitors (e.g., antibodies, particularly monoclonal antibodies, immunologically specific for CTLA-4 such as ipilimumab (Yervoy®)).

The term “wound healing agent” refers to any substance that facilitates the wound healing process. Examples of wound-healing agents include agents that can orient the tissue environment from scarring to regenerative healing by increasing levels of HIF (e.g., a HIF-½ α activator, a master regulator of tissue regeneration. Ciclopirox olamine (Loprox®) is an activator of HIF-α. Interestingly, ciclopirox olamine is also approved for use by the FDA as a topical anti-fungal agent. The antifungal efficacy of ciclopirox may stem from its metal-chelating activity (e.g., iron) given that the actual mechanism of its action has not been proven definitively. Other HIF activators include, without limitation: deferoxamine (e.g., desferoxamine mesylate, desferal), deferoxamine, 3,4-dihydroxybenzoic acid, 1,4-dihydrophenonthrolin-4-one-3-Carboxylic acid (1,4-DPCA) nitric-oxide donor drugs, plant alkaloids (e.g., vinblastine, colchicine), certain phytochemical flavanoids (e.g., quercetin), and oils from tea and other natural-product extracts, as well as known PHD inhibitor now (in 2018) in clinical trials for end-stage kidney failure that act as EPO secretagogues. Some of them work by substrate competition or metal chelation, or both, to inhibit PHD or other enzymes that regulate HIF-α levels in a particular embodiment, the wound healing agent is ciclopirox.

The amount of each component (e.g., antifungal, antiseptic, wound healing agent, immunomodulatory agent, penetrant, antimicrobial, etc.) in the compositions should be great enough for the desired therapeutic effect but low enough to mitigate any undesired side effects. In a certain embodiments, an antimicrobial (e.g., pan-antiseptic) component of the instant compositions may be between about 0.05% (w/v) to about 20.0%, about 0.1% to about 10%, about 0.1% to about 7.5%, about 0.1% to about 5.0%, or about 0.5% to about 5.0%. In a certain embodiments, ethyl pyruvate is present in the compositions from about 1% (w/v) to about 50%, about 5% to about 40%, about 10% to about 30%, about 10% to about 20%, about 15% to about 20%, or about 15% to about 25%. In a certain embodiments, urea is present in the compositions from about 1% to about 40%, about 5% to about 40%, about 10% to about 30%, or about 15% to about 25%.

In a particular embodiment, the antifungal is ciclopirox olamine (0.8%-8.0%) and the antimicrobial is a pan-disinfectant, particularly iodine or chlorhexidine. The composition may further comprise an immunomodulatory agent, particularly an IDO inhibitor such as D-tryptophan, 1-methyl-tryptophan, or methylthiohydantoin-tryptophan. Given that ciclopirox is a divalent cation chelator, including iron, and that iron is a co-factor for both HIF-PHD and IDO enzymes, ciclopirox can play a poly-pharmacological role by promoting wound healing (HIF-α activator) and immune function (IDO inhibitor), in addition to its designated anti-fungal action, per se. The composition may further comprise an active penetrant, including ethyl pyruvate or salicic acid having ketatolytic activity, or a passive penetrant like DMSO (which does not have keratolytic activity in the absence of KOH). In cases where the nail is debrided, alcohols may also serve as passive penetrating solvents.

The composition may further comprise an immunomodulatory agent, particularly an IDO inhibitor such as D-tryptophan, 1-methyl-D-tryptophan, or methylthiohydantoin-DL-tryptophan. The composition may further comprise a wound healing agent, particularly an HIF½a activator such as ciclopirox. Each component of the composition may be present, for example, from about 0.1% (w/v) to about 10%, particularly about 0.5% to about 5% or about 1% to about 5%. The carrier of the composition may be, for example, an aqueous nail lacquer.

In a particular embodiment, the composition comprises ciclopirox, with or without iodine, chloroxyenol, or chlorhexidine, and ethyl pyruvate in a carrier. In certain embodiments, the composition further comprises an immunomodulatory agent, particularly an IDO pathway inhibitor such as D-tryptophan, 1-methyl-tryptophan or methylthiohydantoin-tryptophan. Each of ciclopirox and a pan-antiseptic (iodine, chloroxyenol, chlorhexidine, hypochlorite, etc.) may be present in the composition, for example, from about 0.1% (w/v) to about 10%, about 0.1% (w/v) to about 1%, about 0.5% to about 5%, or about 1% to about 5%. In certain embodiments, ciclopirox is present in the composition from about 1% (w/v) to about 10%, about 1% to about 5%, about 2% to about 10%, about 5% to about 10%, or about 2% to about 8%. In certain embodiments, ciclopirox is present in the composition at about 0.5% (w/v), about 0.8%, about 1%, about 2%, about 3%, about 4%, about 5%, about 6%, about 7%, about 8%, about 9%, or about 10%. In certain embodiments, ethyl pyruvate is present in the composition from about 1% (w/v) to about 50%, about 5% to about 40%, about 10% to about 30%, or about 15% to about 25%. In certain embodiments, ethyl pyruvate is present in the composition at about 1% (w/v), about 5%, about 10%, about 15%, about 20%, about 25%, about 30%, about 40%, about 45%, or about 50%. The carrier of the composition may be, for example, an aqueous nail lacquer or DMSO, among others. In a particular embodiment, the composition contains from about 0.8 to about 8% (w/v) ciclopirox olamine, from about 1% to about 3% (w/v) iodine, from about 1% to about 3% (w/v) chloroxyenol or chlorhexidine, and from about 20% to about 50% (w/v) ethyl pyruvate. In certain embodiments, the composition contains from about 0.8 to about 8% (w/v) ciclopirox olamine, from about 1% to about 3% (w/v) iodine, and from about 20% to about 50% (w/v) ethyl pyruvate. In certain embodiments, the composition contains about 8% (w/v) ciclopirox olamine, about 1% (w/v) iodine, and about 20% (w/v) ethyl pyruvate. In certain embodiments, the composition contains about 8% (w/v) ciclopirox olamine, and about 20% (w/v) ethyl pyruvate.

In a particular embodiment, the composition comprises iodine and ethyl pyruvate in a carrier. Iodine may be present in the composition, for example, from about 0.1% (w/v) to about 10%, particularly about 0.5% to about 5% or about 1% to about 5%. In certain embodiments, iodine is present at about 0.1% (w/v). In certain embodiments, iodine is present at about 1% (w/v). In certain embodiments, ethyl pyruvate may be present in the composition from about 1% (w/v) to about 50%, about 5% to about 40%, about 10% to about 30%, about 15% to about 25%, or about 15% to about 20%. In certain embodiments, ethyl pyruvate is present at about 15% (w/v). In certain embodiments, ethyl pyruvate is present at about 17% (w/v). In certain embodiments, iodine is present from about 0.1% (w/v) to about 1% and ethyl pyruvate is present at about 17% (w/v). In certain embodiments, iodine is present from about 0.1% (w/v) to about 1% and ethyl pyruvate is present at about 15% (w/v). The carrier of the composition may be, for example, an aqueous nail lacquer or DMSO, among others.

In certain embodiments, the compositions provided herein are used to inhibit, treat, and/or prevent onychomycosis. The composition may be applied directly to the nail or may be applied by an applicator such as a brush, wipe, swab, or roller. The compositions may be made into a wide variety of product types such as, without limitation, liquids, lotions, powders, creams, salves, gels, milky lotions, sticks, sprays (e.g., pump spray), aerosols, ointments, pastes, mousses, and other equivalent forms. In a particular embodiment, the compositions are liquids.

In certain embodiments, the compositions provided herein comprise the components described and at least one carrier. The carrier may be a pharmaceutically acceptable carrier and/or a cosmetically acceptable carrier. The carrier should be capable of being commingled with the components of the instant intention in a manner such that there is no interaction which would substantially reduce the efficacy of the active components of the composition (e.g., for treating onychomycosis). A “carrier” refers to, for example, a diluent, adjuvant, excipient, auxiliary agent or vehicle with which an active agent is administered. Carriers may also include stabilizers, penetration enhancers, chelating agents (e.g., EDTA, EDTA derivatives (e.g., disodium EDTA and dipotassium EDTA), iniferine, lactoferrin, and citric acid), and excipients. Carriers can be or comprise sterile liquids, such as water (may be deionized), alcohol (e.g., ethanol, isopropanol), oils (including those of petroleum, animal, vegetable or synthetic origin, such as peanut oil, soybean oil, mineral oil, sesame oil and the like), and other organic compounds or coploymers. Water or aqueous saline solutions and aqueous dextrose and glycerol solutions may also be employed as carriers. Additional general types of carriers include, without limitation, emulsions (e.g., microemulsions and nanoemulsions), gels (e.g., an aqueous, alcohol, alcohol/water, or oil (e.g., mineral oil) gel using at least one suitable gelling agent (e.g., natural gums, acrylic acid and acrylate polymers and copolymers, cellulose derivatives (e.g., hydroxymethyl cellulose and hydroxypropyl cellulose), and hydrogenated butylene/ethylene/styrene and hydrogenated ethylene/propylene/styrene copolymers), solids (e.g., a wax-based stick, soap bar composition, or powder (e.g., bases such as talc, lactose, starch, and the like), and liposomes (e.g., unilamellar, multilamellar, and paucilamellar liposomes, optionally containing phospholipids).

In a particular embodiment, the carrier is a nail lacquer. A “nail lacquer” (also known as a nail varnish or nail polish) refers to a composition which forms a coat or film on the nail. Typically, a nail lacquer comprises a film-forming, organic polymer in a volatile organic solvent. A common nail lacquer comprises nitrocellulose in butyl acetate or ethyl acetate.

Methods of inhibiting, treating, and/or preventing onychomycosis are also provided herein. The methods comprise administering the composition(s) provided herein to the nail of a subject, particularly a human. The nail may be treated, either debrided or not, multiple times a day (e.g., two times or more) or may be administered daily or less frequently. In a particular embodiment the composition(s) is administered three times a day, twice a day, daily, once every two days, or less frequently. The treatment of onychomycosis may be monitored using visual inspection and the composition may be administered as desired or as needed. Treatment may continue for as long as needed (e.g., for at least one month or for at least several months).

Toxicity and therapeutic efficacy of the particular compositions described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form of administration. Dosage amount and interval may be adjusted individually to levels of the active ingredient which are sufficient to, for example, treat onychomycosis.

In certain embodiments, kits are provided. In a particular embodiment, the kit comprises the composition(s) and, optionally, an applicator for applying the composition(s) to the nail. In a particular embodiment, the composition(s) is present within a contained (e.g., vial) along with the applicator (e.g., brush). For example, the applicator may be affixed to the inside of the cap/lid of the vial. As explained hereinabove, the components may be present within one or more compositions. When multiple compositions are used, the kits may comprise multiple containers for the compositions. At least one of the containers may contain an applicator or the applicator may be provided separately. In a particular embodiment, the container is squeezable and allows for the discharge of its contents through an application tip.

As used herein, the singular forms “a,” “an,” and “the” include plural referents unless the context clearly dictates otherwise.

As used herein, the term “onychomycosis” refers to a fungal infection of the nail plate, matrix, and/or nail bed, also known as Tinea unguium. Examples of fungi which are known to cause onychomycosis include, without limitation: dermatophytes (e.g., Trichophyton rubrum, T. interdigitale, Epidermophyton floccosum, T. violaceum, Microsporum gypseum, T. tonsurans, T. soudanense, T. verrucosum); Candida, (e.g., Candida albicans); and nondermatophyte molds (e.g., Aspergillus species, Scopulariosis brevicaulis, Fusarium species, and Scytalidium).

As used herein, the term “drug-resistant skin infection” refers to any topical infection that does not respond to an existing standard of care antibiotic or antifungal treatment. A “therapeutically effective amount” of a compound or a pharmaceutical composition refers to an amount effective to prevent, inhibit, treat, or lessen the symptoms of a particular disorder or disease. The treatment of onychomycosis herein may refer to curing, treating, inhibiting, relieving, and/or preventing onychomycosis, the symptom of it, or the predisposition towards it.

As used herein, “pharmaceutically acceptable” indicates approval by a regulatory agency of the Federal or a state government or listed in the U.S. Pharmacopeia or other generally recognized pharmacopeia for use in animals, and more particularly in humans.

As used herein, “cosmetically acceptable” refers to entities and compositions that are physiologically tolerable and do not typically produce an allergic or similar untoward reaction when administered to an animal, particularly a human, particularly to the skin or nail. In other words, a “cosmetically acceptable” carrier refers to a carrier that can be used without undue toxicity, irritation, allergic response, and the like. Furthermore, the cosmetically acceptable carrier should be capable of being commingled with the components of the instant intention in a manner such that there is no interaction which would substantially reduce the efficacy of the active components of the composition (e.g., for treating onychomycosis). Protocols and procedures which facilitate formulation of the compositions with cosmetic carriers can be found, for example, in Cosmetics & Toiletries Bench Reference (dir.cosmeticsandtoiletries.com/main/login.html) and International Cosmetic Ingredient Dictionary and Handbook. 16th ed. (2016) The Personal Care Products Council, Washington, DC. Each of the foregoing references being incorporated herein by reference.

As used herein, a “carrier” refers to, for example, a diluent, adjuvant, excipient, auxiliary agent or vehicle with which an active agent is administered. Pharmaceutically acceptable carriers can be sterile liquids, such as water and oils, including those of petroleum, animal, vegetable, or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin. A carrier might in some cases also have pharmacological of biological activity, for example, DMSO.

As used herein, an “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, terms that refer to being “anti” a type of target organism (e.g., antimicrobial, antiviral, antifungal, antibacterial, antiparasitic) refers to having any deleterious effects upon those organisms or their ability to cause symptoms in a host or patient. Examples include, but are not limited to, inhibiting or preventing infection, inhibiting or preventing growth or reproduction, killing of the organism or cells, and/or inhibiting any metabolic activity of the target organism. The term “antimicrobial” refers to any substance or compound that when contacted with a living cell, organism, virus, or other entity capable of replication, results in a reduction of growth, viability, or pathogenicity of that entity.

As used herein, the term “kit” generally refers to a collection of elements that together are suitable for a defined use. In a particular embodiment, a “kit” refers to a collection of containers containing the necessary compositions to carry out the method provided, typically in an arrangement both convenient to the user and which ensures the chemical stability of the compositions. The kit may comprise a delivery system having one or more containers (such as tubes or vials). For example, such delivery systems include systems (e.g., enclosures (e.g., boxes)) that allow for the storage, transport, or delivery of compositions provided herein and/or supporting materials (e.g., instruction material) from one location to another.

As used herein, an “instructional material” includes a publication, a recording, a diagram, or any other medium (including electronic) of expression which can be used to communicate the usefulness of the compositions provided herein for performing a method as provided herein. The instructional material of the kit can be shipped or provided with the kit (e.g., affixed to a container) or can be shipped or provided separately from the kit with the intention that the instructional material and kit be used cooperatively by the recipient.

The following examples are provided to illustrate various embodiments of the present invention. The examples are not intended to limit the invention in any way.

### Example 1: EPIC Formulation (Ethyl Pyruvate+Penlac+Iodine or Chlorhexidine)

Penlac® (ciclopirox olamine) is an approved antifungal topical for fungal nail treatment. Penlac® (ciclopirox olamine) is an approved synthetic hydroxypyrimidine antifungal agent thought to inhibit catalase and peroxidase enzymes in fungi as part of its mechanism of action. It is indicated to treat tinea pedis (athlete's foot) and tinea corporis (ringworm) (El-Gohary (2014) The Cochrane database of systematic reviews 8:CD009992). Ciclopirox olamine is used in nail lacquers for topical treatment of onychomycosis, albeit with poor cure rates. Typically, it is used at a 8.0% concentration for the topical treatment of nail fungus. Notably, it has also been determined that this compound is a chemical inhibitor of the IDOL enzyme, which has been implicated widely in immune suppression in cancer and chronic infections. Thus, one might leverage the efficacy of ciclopirox olamine in fungal nails for its combined actions on IDO blockade and HIF-α stabilization in combination with a pan-antiseptic and a nail penetrant. Iodine is a simple, yet highly effective, broad-spectrum antimicrobial agent used widely as a topical antiseptic in medicine. Iodine is not only efficacious against diverse bacteria and protists, including fungi and fungi spores, but unlike other anti-microbial agents it is not susceptible to drug resistance. While some studies have suggested risks to iodine use, for example to the thyroid gland, recent hospital-based comparative studies that are methodical do not corroborate these suggestions and in fact find iodine to be remarkably safe as well as highly effective (Vermeulen et al. (2010) J. Hosp. Infect., 76:191-199).

While a polymer-based formulation of iodine called povidone-iodine (PVP-I; e.g. Betadine®) has been used widely in the clinic, aqueous iodine is also used commonly on skin and nails (tincture of iodine). Interestingly, recent studies suggest that aqueous iodine (tincture of iodine) may be relatively more effective in microbe killing at concentrations of <1% (e.g., 0.1-1%), which is lower than used traditionally in clinical medicine (˜10% for PVP-I). Indeed, “colorless” or “white” iodine—low concentration tincture of iodine solutions—that are sold over-the-counter to the public has been used as a home remedy for treating nail disorders including nail fungus, although such home remedies are not particularly effective.

Chloroxyenol or chlorhexidine is a broad-spectrum antimicrobial chemical used to control bacteria, algae, fungi and virus. Either is used widely in antiseptics, antibacterial soaps, and wound-cleansing applications. Neither is significantly toxic to humans but at high concentrations can be a mild skin irritant in some individuals. Either is used widely in topical antiseptic products at 1%-4% concentration. There is no medical literature on their use to treat onychomycosis.

Ethyl pyruvate (EP) is an FDA generally regarded as safe substance (GRASS) known to improve cutaneous penetration. It is used widely for topical formulation in over-the-counter agents and cosmetics and as a food additive. EP is a safe excipient compound that is known to improve skin penetration of various skin care products and cosmetics, although not for nail care products. However, EP is known to have keratinolysing properties and nails are also composed of keratins like skin. Notably, it has been shown that EP does not react with molecular iodine under physiological conditions (Bell et al. (1967) Proc. Royal Soc. London, Math. Phys. Sci., 298:178-183). Thus, EP is useful as a formulant to iodine to improve its antiseptic activities in the nail bed, based on the established ability of EP to improve drug penetration where keratin-based structures are the chief barrier to penetration.

EP also has immunostimulatory properties, based on its ability to block the immunosuppressive enzyme indoleamine 2,3-dioxygenase (IDO) (Muller et al. (2010) Cancer Res., 70:1845-1853). Thus, EP also helps relieve local immune tolerance of infections, including MMB infections.

In summary, 1% iodine or 3% chlorhexidine and 20% EP is formulated with or without 8.0% ciclopirox olamine in an aqueous solution to treat topical skin infections, or in a standard nail lacquer formulation to treat nail infections, to generate the medicinal cocktail termed EPIC (Ethyl-pyruvate+Penlac+Iodine or Chlorhexidine).

### Example 2: Ciclopirox Activity

A clinical study is performed to evaluate the effectiveness of an innovative topical formulation for killing the pathogens while boosting the patient's immune response to accelerate clearing of the infection. This approach is termed reflexive anti-infective drug synergy (RAIDS) for treating onychomycosis.

A 2-arm blinded and controlled clinical trial on a total of 40 onychomycosis patients, with 20 patients in each patient cohort, is performed to compare the following two topical treatments:
A. Penlac (ciclopirox olamine or ‘ciclopirox’) as a standard of care onychomycosis drug (Control Arm);
B. Penlac plus 20% ethyl pyruvate (EP), which we discovered has immune-enhancing properties, and the pan-antiseptic iodine at 1% (EPIC—Test Arm).

It is well known that ciclopirox is a weak antifungal drug. Our data also suggests that ciclopirox has more potent activities on two signaling pathways that mediate innate immunity: namely, indoleamine dioxygenase-1 (IDO1) and hypoxia inducing factor-1 (HIF1).

i. Ciclopirox Enhances HIF1 Levels

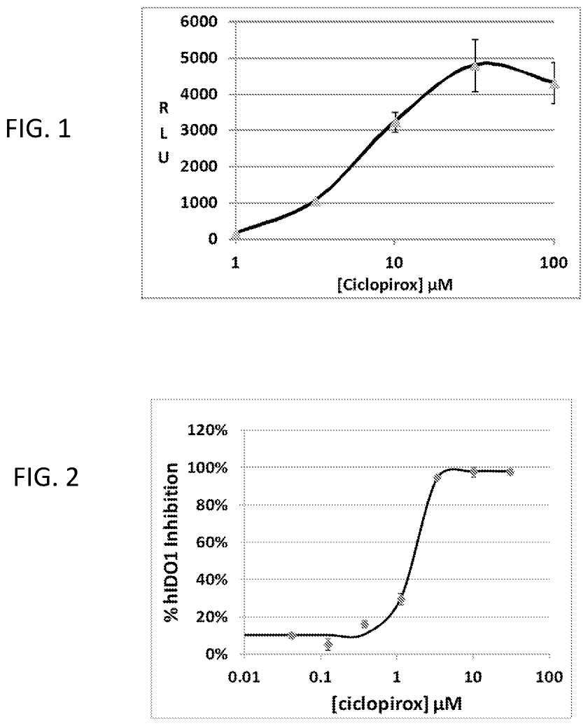

In the course of studies of inhibitors of proline hydroxylase-domain protein (PHD), an enzyme regulating cellular HIF1 levels, we observed that 10 μM ciclopirox enhanced the readout by 15-fold (screening data not shown). We confirmed these findings in a follow-up study and determined that ciclopirox maximally stimulated HIF levels by ˜35-fold over the solvent control, with an apparent EC50 of 5 μM (FIG. 1). The details for the assay protocol are published elsewhere [23].

ii. Ciclopirox Inhibits IDO1 Enzymatic Activity

The IDOL enzyme was first recognized as an antimicrobial [25,26]. The approved topical formulant ethyl pyruvate (EP) can also blunt IDO levels and reverse immunosuppression in the setting of cancer to enable immune eradication [14]. IDO is an iron-dependent enzyme, and we demonstrated that ciclopirox can also act as an IDO enzyme inhibitor. We found that ciclopirox inhibited IDO in a cellular assay with an IC50 of ˜2 (FIG. 2). This is finding is not previously reported in the literature. The details of the assay protocol are published elsewhere [27].

At the time of Penlac's approval as the first FDA-approved topical drug for onychomycosis in 2000, scientists were just beginning to understand the impact of these pathways on innate immunity. The drug's weak intrinsic activity as an antifungal is insufficient to achieve a clinical cure in the vast majority of cases, particularly if onychomycosis is, in fact, a mixed ecology of fungal and bacterial microbes in drug-resistant patients. We designed the EPIC formulation to improve the efficacy of treatments for onychomycosis.

### Onychomycosis is a Polymicrobial Infection

Preliminary data indicate that nails from eight patients diagnosed with onychomycosis (by KOH positive test) are co-infected with bacteria based on DNA testing. Because the drug-resistant nature of onychomycosis might also be due to a local microenvironment in the nail that is immunosuppressed, preventing clearance of the infection, we developed a formulation of EP as an immune enhancing agent, plus iodine as a pan-antiseptic, which is effective against broad pathogen classes.

Others have reported the average MIC concentration for ciclopirox required to kill seven strains of Trichophyton rubrum—the most common dermatophyte in ˜¾ of all onychomycosis patients—as ˜16 μg/ml or 77 μM [28]. This is 10-30 fold higher than the potency of ciclopirox for IDO inhibition and HIF activation, respectively. The findings suggest that the apparent weak antifungal activity of ciclopirox is due to its actions in stimulating innate immune responses via HIF activation (FIG. 1) and IDO inhibition (FIG. 2), respectively—and not due to direct actions for inhibiting fungi alone.

### Clinical Study Design

Only top-level study parameters are summarized below:

| Inclusion Criteria: | Exclusion Criteria: |
|---|---|
| Healthy men and women 50-70 | Known sensitivity to any treatment |
| years old, having ≥20% clinical | ingredients; |
| involvement of at least one great | Use of antifungal medication within |
| toenail; | the prior three-months; |
| Positive KOH test of the infect- | Physical deformity of the affected |
| ed nail; | nail that could complicate photographic |
| Well-controlled diabetes is OK; | assessment for clinical cure rates; |
| Able to understand the Consent | Any pre-condition (including pregnancy |
| Form. | or nursing) that leads the clinician to |
| | any concern on patient safety. |

Up to 50 patients are enrolled to help ensure that 20 patients in each treatment cohort complete the trial. The treatment duration is up to 48 weeks from the time of first treatment in the podiatrist's office to the last office visit. Dynamic randomization proceeds as patients are enrolled to avoid demographic or medical bias in the treatment arms. The treatments limbs are double-blinded to investigators and patients, until after all data analyses are completed. The inclusion of patients aged 50-70 years is intentionally narrow to avoid variability of response due to age in this relatively small pilot trial. Diagnosis of onychomycosis is confirmed before beginning a treatment regimen. A standard KOH test positively confirms the presence of fungus in nail clippings collected from the infected area; and the KOH test is repeated at weeks 24 and 48 to monitor mycological clearance in the prevailing manner. Patient's nails are prepped by standard procedures used in podiatry practice. The formulations are applied by the patients, instructed to consistently do so b.i.d. Patients maintain a patient diary and record any adverse events.

The primary endpoint of the study is complete clinical and mycological cure, assessed at week 52 (4-weeks after completion of therapy). Complete clinical cure is defined as 0% visible involvement of the target toenail. Mycological cure is a negative KOH test. The primary efficacy endpoint is evaluated by treatment group, and within subgroups based on sex, ethnicity and median percent affected toenail area (<40% and ≥40%). Clinical cure rates in the three treatment groups are measured as follows: the outline of the nail in photos is separated into eight segments, each representing 12.5% of the nail. If ≥50% of the segment has an area of infection, the entire segment is counted as area of involved nail. In addition to the quantitative photo assessments, the clinician performs assessments visually at each visit and document the findings to monitor response to treatment. Photographic editing software (Photoshop CS3, Adobe Systems, Inc., San Jose, CA) is used to calculate the ratio of affected nail area to total nail area (in pixel units) for each photograph taken during the course of the study.

### Drug Formulations

The ingredients of the EPIC formulation are: I) ethyl pyruvate (EP at 20%); II) 1% iodine (PVP-I; III) 8% ciclopirox; all mixed in the same base as Penlac, which serves as the standard-of-care Control cohort.

I. Ethyl pyruvate is a safe food ingredient. Inclusion of ethyl pyruvate can normalize the suppressed immune microenvironment at the root of the active infections, acting synergistically with ciclopirox [27,32,33].

II. Iodine (povidone) at 1% is a very safe and effective pan-antimicrobial agent. It is very efficacious against diverse gram-positive and gram-negative bacteria and protists, including fungi and fungal spores; but unlike other anti-microbial agents, it is not susceptible to drug resistance. The antimicrobial action of iodine is rapid, even at concentrations less than 1% [34].

II. Penlac® is an FDA-approved antifungal topical treatment for onychomycosis. It has weak clinical efficacy as a monotherapy. We demonstrated that it also has other intriguing pharmacological actions, namely: 1) activation of the hypoxia-induced factor (HIF) pathway, a master mediator of wound healing; and 2) potent inhibition of IDO enzymatic activity, which disinhibits innate immune responses suppressed by proliferating pathogens.

### DNA Analysis of Pathogens

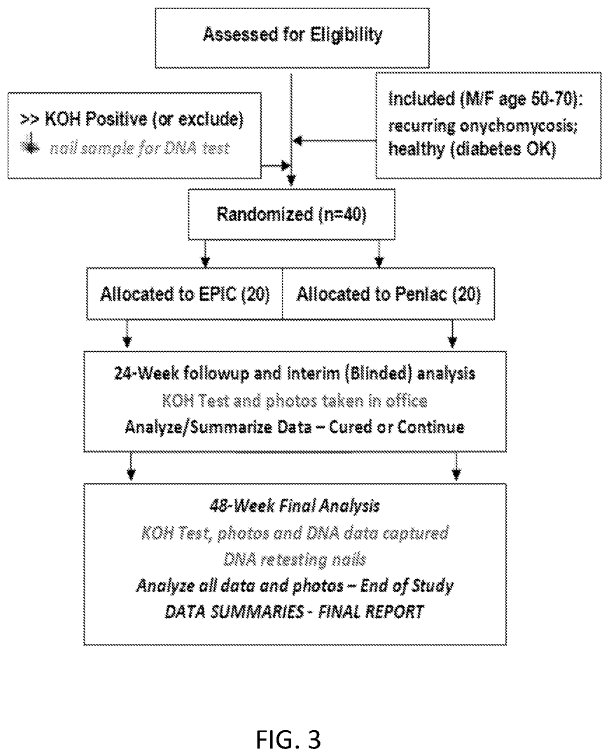

A portion of the nail sample from each patient used for the KOH test and its associated tissue debris is sent to PathoGenius Inc, a CRO in Lubbock, Texas, for DNA analysis of pathogens. The microbial DNA is extracted from the specimen, and then sequenced using next generation platforms to specifically identify fungal and bacterial pathogens. A detailed, statistical report of all pathogens is provided shortly (1-2 weeks) after the samples are received. The approach determines the relative sequence abundance of pathogens in the infected nail sample. This process is accomplished using comparative DNA sequencing analysis, which is widely considered superior to culture analysis for microbial identification. Sequence data are generated by advanced platforms; including Ion Torrent PGM, Ion Proton, and Illumina, and are compared against a sequence database that is curated with updated microbial sequences as they are published. Thus, this approach enables the research team to determine the causative pathogens that represent the disease bioburden of the patient. The data can be used to better understand the outcomes from the clinical study of the EPIC versus ciclopirox formulations (FIG. 3).

### Example 3: Jubliac

Jubliac is formulation which is a combination of efinaconazole (Jublia®) and ciclopirox (Penlac®). The combination has two different mechanisms of antifungal activity. More importantly, the iron-chelating activity of ciclopirox serves to upregulate HIF-α, which promotes wound healing and also inhibit IDO, an effect which enhances immune-aided clearance of the infection. A passive penetrant like DMSO or an active penetrant like EP can be included, among other carriers, including in a nail lacquer.

### Example 4: CicloHex and Ciclo-EP

CicloHex is the combination of ciclopirox for its iron-chelation activity (enhances HIF-α and inhibits IDO—in addition to its antifungal activity) and a pan-antiseptic like chlorhexidine to also quench fungal species.

Ciclo-EP is the combination of ciclopirox and ethyl pyruvate

### Example 5: EPI formulation

A clinical study is performed to evaluate the effectiveness of EPI (Ethyl pyruvate+iodine) and iodine-only to safely treat skin infections or nail infections (onychomycosis). The study is a two-arm design with patients that are randomized to receive nail lacquer formulations that include iodine, a broad-spectrum antimicrobial, with or without ethyl pyruvate, an approved nail penetrant that has been shown to have immunomodulatory properties. The period of treatment is 3 months after recruitment to the study, with follow-up at clinical office visits that are part of standard of care with documentation at 1, 3 and 6 months after the start of treatment.

Rationale for Treatment with Formulations

Nail infections are often difficult to cure. The human nail plate includes three layers, the dorsal and intermediate layers of the nail and the ventral layer comprising the nail bed. The dorsal layer is a few cell layers thick and consists of hard keratin. This layer is the main barrier to diffusion of therapeutics into and through the nail plate. The intermediate layer is also rich in keratin but behaves somewhat differently than the dorsal layer with regard to diffusion. The ventral layer consists of a soft hyponychial where infections take hold beneath the nail plate. Achieving an effective drug concentration in the ventral layer is a key goal of new anti-infective formulations. Chemical penetration through the nail relies upon water solubility and molecular size (Merlin and Lippold, 1997). Penetration enhancers have been studied to increase diffusion of effective chemicals into/through the human nail plate, but agents such as DMSO and isopropyl alcohol that are effective in penetrating skin are either ineffective or impede penetration in the nail. Accordingly, the formulation created for testing in this study includes ethyl pyruvate (EP), a safe approved compound with nail penetrant activity that has been shown to be immunomodulatory.

### EPI Formulation

Iodine is a broad-spectrum antimicrobial agent that kills bacteria, fungi and protists. Notably, unlike other antimicrobial agents, iodine is not susceptible to drug resistance. Low concentrations (<1%) are as potent as higher concentrations used historically in medicine (e.g. ˜10% in PVP-I [Betadine®]). For example, low concentration tincture of iodine is available OTC as ‘white iodine’ and can be used as a home remedy for topical infections. While some studies have suggested risks to iodine use, for example to the thyroid gland, recent hospital-based comparative studies that are methodical do not corroborate these suggestions and in fact find iodine to be remarkably safe as well as highly effective (Vermeulen et al., 2010).

Ethyl pyruvate (EP) is widely used as a pharmaceutical excipient (formulant) to improve skin penetration of various skin care products and cosmetics. It is defined by the FDA as a generally regarded as safe substance (GRASS). In considering its co-formulation with iodine, studies have shown that EP does not react with molecular iodine under physiological conditions (Bell and Ridgewell, 1967). Notably, it was discovered that EP can stimulate immunity by blocking expression of indoleamine 2,3-dioxygenase (IDO) (Muller et al., 2010), an immunosuppressive enzyme elevated in certain chronic skin infections (e.g. warts (Xie et al., 2017)). Thus, EP can improve the antiseptic properties of iodine by two mechanisms, one through improving nail bed penetration and another by relieving immune tolerance to MMB infections such as onychomycosis.

### Study Endpoints

The primary endpoints of the study are to determine the nail infection status after treatment at 1, 3 and 6-month visits, documenting the final status or absence of infection in all patients.

GMP formulations

### EPI Formulations

1) Aqueous solution of iodine (0.1-1% w/v) and EP (17% w/v) in a standard nail lacquer formulation.
2) Aqueous solution of iodine (1% w/v) and EP (15% w/v) in a standard nail lacquer formulation

USP standards for EP grade do not exist (only food grade standards). Thus, the EP employed in the EPI formulation is obtained commercially as the highest-grade material available (e.g. analytic grade, which is >98% pure). Similarly, iodine is not subject to USP standards and is obtained for use at highest grade suitable for human use. Iodine as included at 0.1 or 1% also serves a preservative to the EPI solution.

Safety: EP is a stable and metabolizable derivative of pyruvate that has been studied in animals and humans where no evidence of significant toxicities have been reported (Fink, 2007; Kao and Fink, 2010). In humans, EP has been delivered by i.v. infusion of >50% solutions where toxicity issues as part of an IND application were addressed in support of a Phase I/II trial in cardiac surgery (http://clinicaltrials.govict2/show/NCT00107666). No significant toxicities were reported in this study which also failed to reveal any therapeutic benefit (Bennett-Guerrero et al., 2009). In the clinical setting, other than small studies of Vircin®, no evidence exists that as a topical agent EP serves as anything other than a benign GRASS excipient. In summary, in what appears to be extensive experience with EP in preclinical, clinical, food industry and cosmetic settings, whether administered topically, orally, or systemically (intravenously) over a wide range of concentrations, the available information suggests that the safety risks of EP as a formulation excipient are extremely low.

### Iodine Considerations

OTC formulations available as “tincture of iodine” are low-concentration preparations 0.1-1% iodine, sometimes known informally as “colorless” or “white” iodine. Tincture of iodine has been used for nail treatment as a home remedy. While over the years some studies have suggested that the topical use of iodine may involve limited safety risks, more methodical hospital-based studies do not corroborate these suggestions and instead find iodine to be very safe as well as effective (Vermeulen et al., 2010). Given the long history of experience with iodine as topical antiseptics, the risk-benefit ratio from their use is argued to be extremely low.

### Aims

Aim 1. Record the clinical status of the nail infection of patients at the start of the study and at each follow up visit (i.e. diagnosis, extent of infection visible, number of nails infected)

Aim 2. Record the standard-of-care treatment provided to the patient at the first study visit, if any. At the same visit where informed consent is obtained, or within two weeks of consent, all patients who participate in the study receive clinical standard of care for nail infections that may include topical debridement or removal of part of the nail. These treatments are variably employed at the discretion of the clinical investigators. Diagnosis, location, size and number of infections are recorded along with the type of care provided per clinical standards.

Aim 3. Randomize patients to the EPI or I-only arms of the trial, providing treatment at the first study visit. At the end of the first visit, patients are randomized into two cohorts constituting the EPI or I-only groups. Patients are provided with a vial containing each formulation. Each vial is labeled ‘For Nail Treatment’ with the one-sentence instruction ‘Apply the treatment to fingernails twice a day for three months’. These instructions are confirmed verbally by the study coordinator before the patient leaves the first study visit.

Aim 4. Receive nail photographs taken bimonthly by patients on their mobile phone and emailed to the clinical coordinator. Every two weeks, patients take mobile phone photographs of the back of the hand close enough to show the status of their fingernails, and then email the photos to the clinical coordinator. These instructions are confirmed verbally before the patient leaves the study visit.

Aim 5. Record the clinical status of the nail infection(s) on the patient, if any, indirectly from photos every two weeks and directly at follow-up visits at the end of months 1, 3 and 6 (study endpoint). Patients are examined one month after the first study visit to assess the outcome of their first treatment month. Nail status and care, if provided, are recorded at the time of care during this second study visit. The same exam is performed at months 3 and 6. A biostatistician serves as a consultant for comparative interpretation of the clinical observations.

### Study Timeline

Total estimated time for this study is 24 months.
Phase 1: Finalize protocol (months 0-3).
Phase 2: Recruit subjects (months 4-15).
Phase 3: Perform subject follow-up (months 16-19).
Phase 5: Conduct data analysis and prepare communications (months 20-24).

### Example 6: Treatment of a Skin Infection

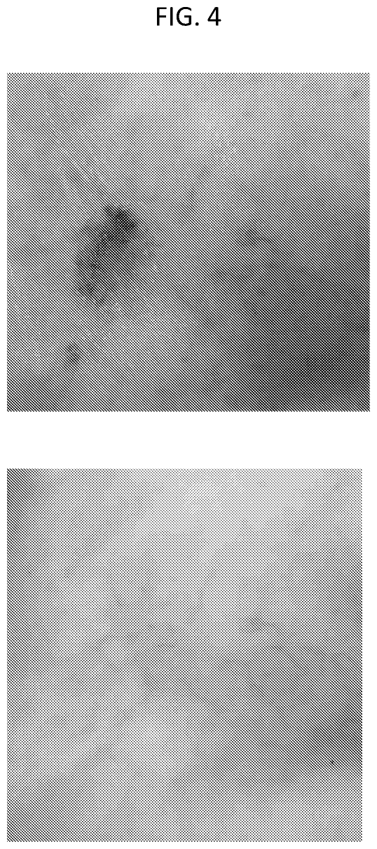

EPI and EPIC formulations are suitable for topical treatment of skin infections. For example, a patient having a skin Staphylococcus infection on the foot was treated with an EPI formulation. Treatment was administered topically daily for one week. The patient received no other treatment for the infection. FIG. 4 shows clinicopathology photos taken before (top) and after (bottom) treatment.

### REFERENCES

1 Del Rosso, J. Q. (2014) The role of topical antifungal therapy for onychomycosis and the emergence of newer agents. J Clin Aesthet Dermatol 7 (7), 10-18
2 Tverdek, F. P. et al. (2016) Antifungal agents and liver toxicity: a complex interaction. Expert Rev Anti Infect Ther 14 (8), 765-776
3 Tosti, A. and Elewski, B. E. (2016) Onychomycosis: Practical Approaches to Minimize Relapse and Recurrence. Skin Appendage Disorders 2 (1-2), 83-87
4 Rich, P. (1996) Special patient populations: onychomycosis in the diabetic patient. J Am Acad Dermatol 35 (3 Pt 2), S10-12
5 Ghannoum, M. and Isham, N. (2014) Fungal Nail Infections (Onychomycosis): A Never-Ending Story? PLoS Pathogens 10 (6), e1004105
6 Westerberg, D. P. and Voyack, M. J. (2013) Onychomycosis: Current trends in diagnosis and treatment. Am Fam Physician 88 (11), 762-770
7 Rosen, T. et al. (2015) Onychomycosis: epidemiology, diagnosis, and treatment in a changing landscape. J Drugs Dermatol 14 (3), 223-233
8 Elewski, B. E. (1998) Onychomycosis: Pathogenesis, Diagnosis, and Management. Clinical Microbiology Reviews 11 (3), 415-429
9 Niewerth, M. et al. (2003) Ciclopirox olamine treatment affects the expression pattern of Candida albicans genes encoding virulence factors, iron metabolism proteins, and drug resistance factors. Antimicrob Agents Chemother 47 (6), 1805-1817
10 Piraccini, B. M. et al. (2010) Long-term follow-up of toenail onychomycosis caused by dermatophytes after successful treatment with systemic antifungal agents. J Am Acad Dermatol 62 (3), 411-414
11 Gellin, B. et al. (2001) Adjunctive Immune Therapy for Fungal Infections. Clinical Infectious Diseases 33 (7), 1048-1056
12 Zinkernagel, A. S. et al. (2007) Hypoxia inducible factor (HIF) function in innate immunity and infection. J Mot Med (Berl) 85 (12), 1339-1346
13 Schaffer, K. and Taylor, C. T. (2015) The impact of hypoxia on bacterial infection. Febs j 282 (12), 2260-2266
14 Muller, A. J. et al. (2010) Immunotherapeutic suppression of IDO and tumor growth with ethyl pyruvate. Cancer research 70 (5), 1845-1853
15 Emens, L. A. et al. (2016) Cancer immunotherapy trials: leading a paradigm shift in drug development. J Immunother Cancer 4, 42
16 Messerschmidt, J. L. et al. (2016) How Cancers Escape Immune Destruction and Mechanisms of Action for the New Significantly Active Immune Therapies: Helping Nonimmunologists Decipher Recent Advances. Oncologist 21 (2), 233-243
17 Harding, M. W. et al. (2009) Can filamentous fungi form biofilms? Trends Microbiol 17 (11), 475-480
18 Desai, J. V. et al. (2014) Fungal biofilms, drug resistance, and recurrent infection. Cold Spring Harb Perspect Med 4 (10)
19 Nusbaum, A. G. et al. (2012) Biofilms in dermatology. Skin Therapy Lett 17 (7), 1-5
20 Rhoads, D. D. et al. (2012) Clinical identification of bacteria in human chronic wound infections: culturing vs. 16S ribosomal DNA sequencing. BMC Infect Dis 12, 321
21 Dowd, S. E. et al. (2011) Survey of fungi and yeast in polymicrobial infections in chronic wounds. J Wound Care 20 (1), 40-47
22 Wolcott, R. D. et al. (2010) Healing and healing rates of chronic wounds in the age of molecular pathogen diagnostics. J Wound Care 19 (7), 272-278, 280-271
23 Zhang, Y. et al. (2015) Drug-induced regeneration in adult mice. Science Translational Medicine 7 (290), 290ra292-290ra292
24 Linden, T. et al. (2003) The antimycotic ciclopirox olamine induces HIF-1alpha stability, VEGF expression, and angiogenesis. Faseb j 17 (6), 761-763
25 Divanovic, S. et al. (2011) Opposing Biological Functions of Tryptophan Catabolizing Enzymes During Intracellular Infection. The Journal of Infectious Diseases 205 (1), 152-161
26 Pfefferkorn, E. R. (1984) Interferon gamma blocks the growth of Toxoplasma gondii in human fibroblasts by inducing the host cells to degrade tryptophan. Proc Natl Acad Sci USA 81 (3), 908-912
27 Muller, A. J. et al. (2010) Immunotherapeutic suppression of indoleamine 2,3-dioxygenase and tumor growth with ethyl pyruvate. Cancer Res 70 (5), 1845-1853
28 Schaller, M. et al. (2009) Susceptibility testing of amorolfine, bifonazole and ciclopiroxolamine against Trichophyton rubrum in an in vitro model of dermatophyte nail infection. Medical Mycology 47 (7), 753-758
29 Lai, Y. W. et al. (2016) Synergy and antagonism between iron chelators and antifungal drugs in Cryptococcus. Int J Antimicrob Agents 48 (4), 388-394
30 Bruhn, K. W. and Spellberg, B. (2015) Transferrin-mediated iron sequestration as a novel therapy for bacterial and fungal infections. Curr Opin Microbiol 27, 57-61
31 Kim, J. et al. (2012) A defect in iron uptake enhances the susceptibility of Cryptococcus neoformans to azole antifungal drugs. Fungal Genet Biol 49 (11), 955-966
32 Mellor, A. L. and Munn, D. H. (2008) Creating immune privilege: active local suppression that benefits friends, but protects foes. Nat Rev Immunol 8 (1), 74-80
33 Goldszmid, R. S. et al. (2014) Host immune response to infection and cancer: unexpected commonalities. Cell Host Microbe 15 (3), 295-305
34 Sokal, D. C. and Hermonat, P. L. (1995) Inactivation of papillomavirus by low concentrations of povidone-iodine. Sex Transm Dis 22 (1), 22-24
35 Vermeulen, H. et al. (2010) Benefit and harm of iodine in wound care: a systematic review. J Hosp Infect 76 (3), 191-199
36 Lee, S. K. et al. (2005) Allergic contact dermatitis from iodine preparations: a conundrum. Contact Dermatitis 52 (4), 184-187
37. Bennett-Guerrero, E. et al. (2009) A phase II multicenter double-blind placebo-controlled study of ethyl pyruvate in high-risk patients undergoing cardiac surgery with cardiopulmonary bypass. J Cardiothorac Vasc Anesth 23 (3), 324-329
38. Vestby, L. K. and Nesse, L. L. (2015) Wound care antiseptics—performance differences against Staphylococcus aureus in biofilm. Acta Veterinaria Scandinavica 57 (1),

Several publications and patent documents are cited in the foregoing specification in order to more fully describe the state of the art to which this invention pertains. The disclosure of each of these citations is incorporated by reference herein. US Provisional Patent Application No. 63/162,675, filed Mar. 18, 2021, and US Provisional Patent Application No. 63/177,718, filed Apr. 21, 2021, are incorporated by reference herein. While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A composition comprising:
a) at least one antimicrobial; and
b) at least one immunomodulatory agent.

2. The composition according to claim 1, wherein the antimicrobial is the antifungal ciclopirox, which also serves to enhance immune function and wound healing, via HIF-α stabilization.

3. The composition according to claim 1, wherein the antimicrobial is the pan-antiseptic iodine or chlorhexidine.

4. The composition according to claim 1, wherein the immunomodulatory agent is an indoleamine dioxygenase (IDO) inhibitor.

5. The composition according to claim 4, wherein the IDO inhibitor is D-tryptophan, 1-methyl-tryptophan, or methylthiohydantoin-tryptophan, or ethyl pyruvate.

6. The composition according to claim 1, wherein the composition further comprises a carrier.

7. The composition according to claim 6, wherein the carrier is ethyl pyruvate or DMSO.

8. The composition according to claim 1, wherein the composition further comprises a penetrant.

9. The composition according to claim 8, wherein the penetrant is ethyl pyruvate or DMSO.

10. The composition according to claim 1, wherein the composition further comprises a wound healing agent.

11. The composition according to claim 10, wherein the wound healing agent is an HIF-α activator.

12. The composition according to claim 11, wherein the HIF-α activator is ciclopirox.

13. The composition according to claim 1, further comprising a carrier that is a nail lacquer.

14. The composition according to claim 1, comprising iodine or chlorhexidine, and ethyl pyruvate.

15. The composition according to claim 14 comprising
(a) about 1% (w/v) iodine or about 3% (w/v) chlorhexidine, and/or about 20% (w/v) ethyl pyruvate;
(b) about 0.1% (w/v) to about 1.0% (w/v) iodine and/or about 17% (w/v) ethyl pyruvate; or
(c) about 1.0% (w/v) iodine and/or about 15% (w/v) ethyl pyruvate.

16.-17. (canceled)

18. The composition according to claim 1, comprising ciclopirox, iodine or chlorhexidine, and ethyl pyruvate.

19. The composition according to claim 18, comprising about 8% (w/v) ciclopirox, about 1% (w/v) iodine or about 3% (w/v) chlorhexidine, and/or about 20% (w/v) ethyl pyruvate.

20. A method of inhibiting, treating, and/or preventing a topical infection in a subject, wherein the method comprises administering topically the composition according to claim 1 to the subject.

21. The method according to claim 20, wherein the topical infection is onychomycosis or a skin infection.

22.-26. (canceled)

27. A kit comprising the composition according to claim 1 and, optionally, an applicator.
